(19) United States
(12) Patent Application Publication
(10) Pub. No.: US 2004/0243439 A1
(43) Pub. Date: Dec. 2, 2004

(54) METHODS AND SYSTEMS FOR CLINICAL TRIAL DATA GATHERING AND MANAGEMENT

(75) Inventors: Arthur G. Huggard, Wildwood, MO (US); John D. Sanford, Pensacola Beach, FL (US)

Correspondence Address: BLACKWELL SANDERS PEPER MARTIN LLP, 720 OLIVE STREET, SUITE 2400, ST. LOUIS, MO, 63101, US

(73) Assignee: SOLUTIA INC., St. Louis, MO (US)

(21) Appl. No.: 10/709,756

(22) Filed: May 26, 2004

Related U.S. Application Data
(60) Provisional application No. 60/474,455, filed on May 30, 2003.

(51) Int. Cl. G06F 17/60; A61B 5/00

(52) U.S. Cl. 705/2; 715/513

(57) ABSTRACT

Methods and associated structure for automating clinical trial data gathering and management in the pharmaceuticals development industry. Clinical trial data is entered directly by examining clinicians through use of a remote computing system such as a handheld device, laptop computer or tablet PC. The system permits direct data entry by the clinician both as electronic form data that may be locally validated by the remote computing device as well as digitized images of handwritten or hand-drawn information. The remote computing device may forward the validated clinical data to a central servers system using secured communication and storage techniques. When the remote computing device is without communications to the central server, such as may be the case occasionally using wireless communication links, secure storage within the remote computing device may spool entered information in a secured local storage until such time as secured communications is re-established with the central server.

### CROSS REFERENCE TO RELATED APPLICATIONS

This application claims the benefit of U.S. Provisional Application Ser. No. 60/474,455 Filed May 30, 2003.

### BACKGROUND OF INVENTION

In pharmaceutical development, one critical aspect of the process involves performing clinical trials of a proposed new pharmaceutical or medical device in preparation for regulatory approval. Such clinical trials involve usage of the proposed new pharmaceutical or medical device on a large number of patients and monitoring of results and potential side effects of such usage in each patient. Such clinical trials are done in phases and span a significant period of time. Timely and accurate collection and management of such clinical trial data has presented an on going problem for the industry.

In general, even at present, most clinical trial data gathering and management involves substantial manual processing. A clinician (i.e., physician, investigator, medical designee, or other health care personnel) must first determine if a patient is a candidate for a clinical trial and if so complete an enrollment process with the organization managing the trial. (often referred to as the Contract Research Organization or CRO but this could also be a CRO like group at the company or organization sponsoring the research; also known as the sponsor. For purposes of this patent, we use “Data Service Provider” to refer to any organization managing the clinical trial). Once enrolled, a series of visits, treatments and testing is set up for the patient which must be closely monitored and documented. The clinician gathers data associated with examination of the patient at various stages of the clinical trial process. The clinician generally prepares handwritten notes and documents indicating results of the examination. Often the information is provided by filling out a form requesting standardized data regarding the clinical trial. Such information is then forwarded in a secure manner to the Data Service Provider for entry, data validation and analysis. Typically the papers produced by the clinician are faxed or mailed to the data entry service provider. Such data entry service providers receive clinical trial data from various clinicians and enter the data into a centralized server database for further analysis. Due to the critical nature of the data in regulatory processing, the data is often entered twice and then cross-checked to verify its accuracy with respect to the original handwritten or drawn information. Data so entered and validated in the central server database may then be used to perform required analysis and to produce required regulatory information for submissions to regulatory agencies. Any errors are referred to as Queries and must be resolved with the clinician. Paper based entry systems are subject to many queries as handwriting and terminology may differ among clinicians.

At all stages of such processes, data security is critical due to the sensitive nature of the information. The information is sensitive with respect to patient confidentiality as well as pharmaceutical industry competition. Paper documents and reports produced by the clinicians' examination must be physically secured as required by regulatory agencies. Paper documents are faxed or mailed to the service provided as a secure means of communication. An authorized data entry service provider then imposes still further physical and electronic security measures on the data received and entered.

Although data entry and management automated systems have advanced, the clinical trial data gathering and management industry has been slow to adopt such new technologies. A number of factors may explain reluctance to adopt new technologies. First, the new technologies have not adapted to provide a friendly user interface for a typical clinician. For example, present clinical trial data collection and management systems do not permit a clinician to easily enter handwritten or drawn annotations to an examination report. Still further, the need for high security with respect to the gathered data and the need for auditable verification of the process from the clinician through various intermediates to the final central system database still imposes a significant burden on the clinician. At best, present electronic or web based systems may have moved the data re-keying process into the clinician's office where his/her staff may re-enter the clinician's data from notes taken in the patient exam. This process continues to involve manual steps to transfer data from the clinician to associated staff people. Maintaining such manual procedures as presently practiced imposes delays and hence cost on the process of clinical trial data gathering and management. These additional costs and delays may ripple into other costs and delays in the pharmaceutical development and regulatory approval process.

It therefore remains an ongoing problem for the pharmaceuticals industry to gather and manage clinical trial data in such a manner to assure required security and accuracy while at the same time reducing the need for manual intervention and hence improving productivity, speed and accuracy in entry of the data.

### SUMMARY OF INVENTION

The present invention solves the above and other problems, thereby advancing the state of the useful arts, through methods and associated structures for more fully automating clinical data gathering and management processes. Features and aspects hereof provide for secure data entry and storage in all electronic form directly from the examining clinician. In one specific aspect hereof, a remote computing device (such as a tablet PC, laptop computer or other handheld remote computing device) in the hands of the examining clinician permits direct, electronic data entry from the clinician to the clinical data management systems. Such a remote computing device may permit entry of data both as “fill in the blanks” electronic forms as well as digitized images of handwritten notes or hand drawn figures. Form data entered at such a remote computing device may be locally validated by processing power local to the remote computing device. Handwritten notes and figures may also be saved as digitized images to be forwarded to the central server database.

In another aspect hereof, electronic data entered by a clinician on the remote computing device maybe securely stored locally during periods of time that the remote computing device is without communications to a central server. When the remote computing device again establishes communication to the central server, data may be transmitted thereto immediately following local validation. When the remote computing device is temporarily without communication capabilities to the central server, security features hereof permit the data to be locally validated and stored secured from unauthorized access until such time as communications with the central server is again established. These and other aspects hereof also permit wireless communications to be utilized between the remote computing device and the centralized data management server, although wireless communication capability is not a requirement. By allowing the clinician an interface that simplifies direct data entry, intermediate steps requiring the manual re-keying of data by a data entry service provider may be eliminated thus improving both accuracy of the gathered data and speed in the gathering of such data.

### BRIEF DESCRIPTION OF DRAWINGS

FIG. 1 is a block diagram of an exemplary system in which features and aspects hereof may be advantageously applied.

FIG. 2 is a flowchart describing a method for clinical trial data collection at a remote computing device.

FIG. 3 is a flowchart describing a method for receiving and archiving clinical trial data within a central server.

FIG. 4 is a flowchart describing a method for reporting clinical trial data archived in a central server.

### DETAILED DESCRIPTION

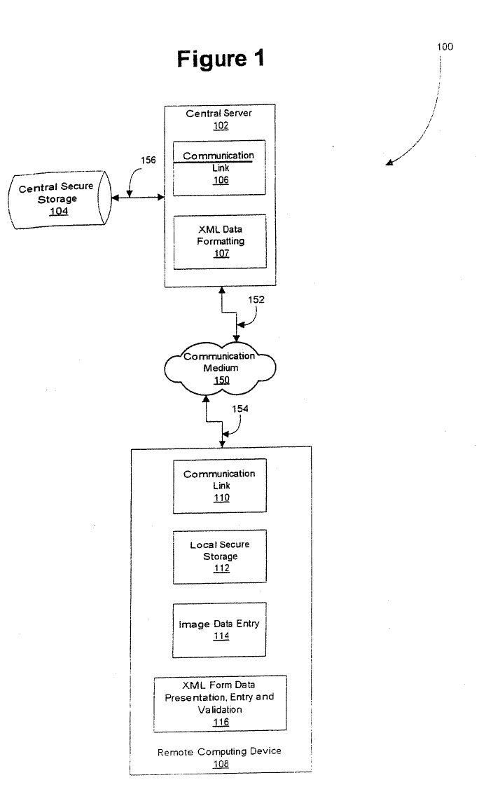

FIG. 1 is a block diagram of a system 100 embodying features and aspects hereof. Remote computing device 108 interacts with a clinician examining the patient participating in a clinical trial study. The clinician may enter data on remote computing device 108 either as handwritten or hand drawn images or as data entered into fields of a form, or both. Such a form may correspond to standardized information required in the clinical trial study and may therefore prompt the clinician to respond with the requested information. Image data entry element 114 may receive such handwritten or hand drawn information and digitizes such information to be stored in association with form data required in the clinical trial study. Form data presentation, entry and the validation element 116 is operable to present form data to a clinician, to receive clinician responses for the requested information, and to validate the clinicians entered responses. In one aspect hereof, element 116 may present the user with a form to be filled in accordance with XML format definitions (i.e., an XML schema). Such an XML schema may define, among other things, the format for presentation of information to the clinician as well as fields for entry of clinician responses. Still further, an XML schema may define validation criteria (i.e., data edits). These validation criteria may be applied locally by operation of the remote computing device 108 to verify the entered data in accord with the XML schema. Further validation of such entered information may also be provided by the central server as discussed further herein below.

Remote computing device 108 may also include a secure local storage medium 112 in which clinical trial data entered it by the clinician may be temporarily stored in a secure manner. As noted above, regulatory requirements for such clinical trial data may include security requirements to prevent unauthorized access to private medical information associated with the clinical trial patient. Still further, corporate security measures imposed within the pharmaceutical development industry may require still stricter security measures to further preclude unauthorized access to the gathered clinical trial data. Local secure storage element 112 may be implemented as any of several well known storage media including, for example, nonvolatile semiconductor memory components (i.e., PC card memory modules or flash memory modules), magnetic disk or tape storage media, optical disk or tape storage media, etc. Security for such a storage media may be provided in a number of forms depending on regulatory and corporate security requirements. In one aspect hereof, security may be provided as client certificates providing digital encryption techniques using public and/or private key encryption. In one example, such an encryption key may be generated as a function of a supplied user ID and password. Numerous other digital encryption techniques will be readily apparent to those of ordinary skill in the art. All such encryption techniques may be useful for securing the storage of sensitive data in local secure storage element 112.

Remote computing device 108 may include communication link 110 adapted for communicating with an associated central server system (i.e., central server 102 of system 100). Communication link 110 may provide, for example, network communication features to couple the remote computing device 108 to a central server using any of numerous well known computer networking techniques. As required in a particular application, communication link 110 may also provide encryption or other security techniques to secure data transmission between the remote computing device 108 and an associated server from unauthorized interception. In one aspect hereof, communication link 110 provides a wireless networking communication feature for coupling the remote computing device to an associated central server 102 without the need for a tethered connection that may restrict less convenient. Such a wireless communication technique further simplifies use of the remote computing device 108 for a non-technical clinician desiring ease of use. Wireless networking communication is not a requirement since data collected offline may be readily and securely posted to the centralized server whenever a hard wired connection is available. Numerous other computer networking connections may be provided by communications link 110 to couple remote computing device 108 to associated central server 102 as required in a particular application.

Remote computing device 108 may be implemented as any remote computer system useful for a particular application including, for example, handheld devices such as laptop or notebook computers, personal digital assistants (PDAs) and tablet PC systems. Features and aspects of the remote computing device 108 may also be provided by less mobile (i.e., desktop) but with some loss of flexibility in the lack of mobility. Ease of use as reflected by the mobility of a handheld remote device is one desirable, though not required, aspect hereof. In one particular aspect hereof, so-called tablet PC system may be used for remote computing device 108. Such a tablet PC provides desired features for local validation and entry of form data, for secure storage of such entered data, and for input of digitized images of handwritten or hand drawn ancillary information from the clinician.

Central server 102 of system 100 generally provides centralized storage and archiving of clinical trial data gathered from one or more remote computing devices 108. Numerous clinicians, each using a remote computing device 108, may enter clinical trial data during examination procedures of each clinic hr study patient. As noted above, such clinical trial data may be initially stored locally within the remote computing device and, when conductivity permits, forwarded at a later time to the central server 102. Central server 102 may therefore include a communication link 106 similar to the communication link 110 described above with respect to remote computing device 108. Communication link 106 may provide network connectivity to one or more remote computing devices 108 as discussed above. In one aspect hereof, communication link 106 provides a wireless network communication link to one or more remote computing devices 108. As further noted above, such wireless communication links enable additional mobility and flexibility for clinicians utilizing remote computing devices to enter data during clinical trial patient examinations.

Central server 102 may further include XML reformatting element 107 to provide desired reformatting of clinical trial data stored by central server 102. Such reformatting may include generation of desired reports useful in the regulatory process as well as other administrative reporting to provide desired statistical summaries and analysis of gathered clinical trial data. Central secure storage 104 may be associated with a central server 102 as a central repository of gathered clinical trial data. Central secure storage 104 may be coupled to central server 102 either locally or remotely via path 156.

As noted above, although significant validation and verification of entered clinical trial data may be provided by remote computing device 108, central server 102 may include within XML data reformatting element 107 the capability of further verification and validation of data provided by an associated remote computing device 108. Where such an additional validation or verification indicates a problem with provided clinical trial data, the data may be returned to the remote computing device with an appropriate error or status indicator to permit correction and reentry of the provided clinical trial data.

As a above with respect to remote computing device 108, central server 102 may apply digital encryption techniques to assure security of any communications with remote computing devices 108 as well as to assure security of clinical trial data stored within central secure storage 104. As above, client/server certificates generated based upon user ID's and/or passwords may be utilized to provide such encryption security. Numerous other well known forms of encryption and security may be applied to assure the desired level of security for communications with remote computing devices and for storage of private clinical trial data.

Central server 102 may be coupled via path 152 to a network communication medium 150. In like manner, remote computing device 108 may be coupled via path 154 to communication medium 155. As discussed above, central server 102 may be coupled thereto through communication link 106 while remote computing device 108 may be coupled with thereto via communication link 110. Communication medium 150 may be any appropriate medium for transmission of clinical trial data between one or more remote computing devices 108 and a central server 102. Such communication media may be, for example, Ethernet, token ring, Fibre Channel (or other optical communication media), as well as wireless communication features utilizing RE or other frequencies of the electromagnetic radiation spectrum. The associated communication link (i.e., 106 and 110) and the communication medium 150 may preferably provide any desired level of security through data encryption or other well known encryption and security techniques.

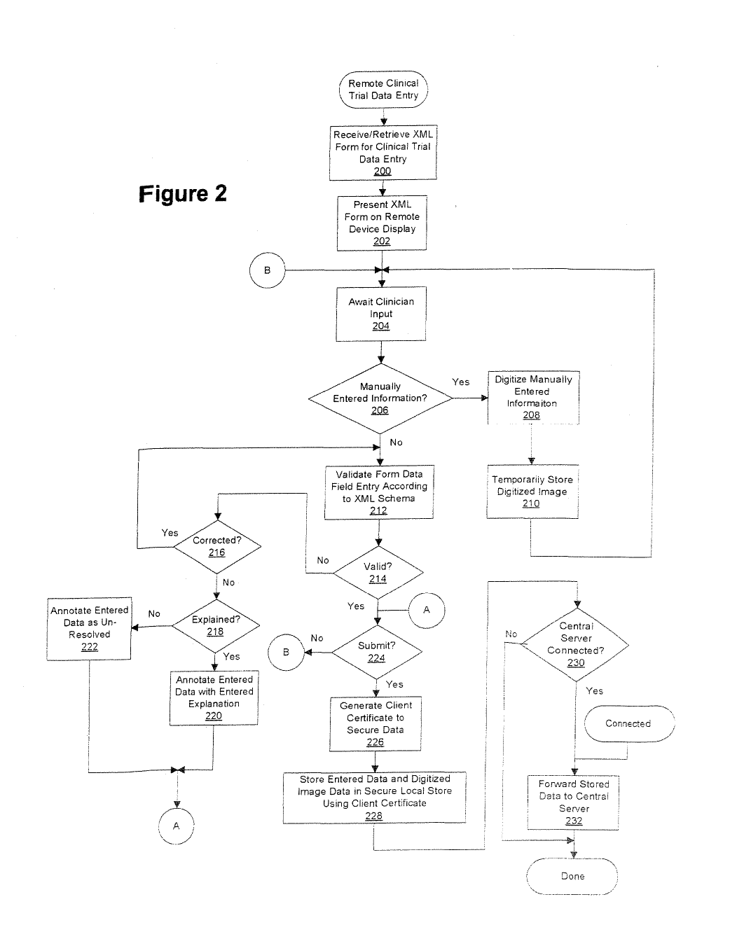

FIGS. 2 through 4 are flowcharts describing processes operable within a system such as described above with respect to FIG. 1. In particular, FIG. 2 describes a process operable within a remote computing device including, for example, PDAs, laptop or notebook computers, tablet PC systems and the like. Element 200 is first operable to receive or otherwise retrieve an XML form designed for clinical trial data entry. The XML form may be previously received from an associated central server system or other repository of XML forms useful for clinical trial data entry. Alternatively, the XML form may be locally stored (i.e., cached) within the remote computing device. Element 202 then presents the XML form on a display associated with the remote computing device. Element 204 next awaits receipt of input from the clinician user of the remote computing device indicating entry of information in some field(s) of the displayed XML form.

If the received clinician input is manually entered information (i.e., handwritten or hand drawn annotations), as determined by element 206, processing continues with element 208 to digitize such manually entered information followed by element 210 to temporarily store the digitized information in the local secure store. Digitizing of such manually entered information may consist of scanning paper documents using a scanner feature associated with the remote computing device or may comprise direct receipt of digitized information as sketched into an electronic digitizing tablet associated with the remote computing device. As noted elsewhere herein, in one aspect of the invention, a tablet PC having such a digitizing surface integrated within may be used for entry of such manual information. Following digitized entry of the information and temporary storage of the digitized image, processing continues with element 204 to await further clinician user input.

If element 206 determines that the received clinician input is not manually entered information but rather entry of data in fields of the form presently displayed, element 212 is next operable to perform validation functions on the data field just entered. Such a validation may be in accordance with information imbedded in the XML schema of the presented form. So called “data edits” may be defined within an XML schema to indicate test criteria which may be applied to the corresponding data entry field to determine whether the entered data complies with expected values or ranges. Numerous types of data edits may be associated with an XML schema as well known to those of ordinary skill in the art. Numerous other validation processes may also be applied where XML schemas are not utilized to define the desired, expected data values and ranges.

If the validation process of element 212 indicates that the data is not valid as determined by element 214, element 216 is next operable to permit the clinician user to correct the erroneous data entry. If the clinician enters corrected information, processing returns to element 212 to again verify correctness of the newly corrected data entry. If the clinician chooses not to enter corrected data as determined by element 216, element 218 allows the clinician to enter an annotation explaining the discrepancy in the entered data field. If the clinician enters such an annotation explaining the discrepancy, element 220 is next operable to annotate the entered data in temporary storage with the provided explanation regarding the validation discrepancy. If no such annotation is entered by the clinician user as determined by element 218, element 222 is operable to annotate the entered data field as an unresolved discrepancy. Such unresolved discrepancies may be resolved at a later time in the clinical trial study. In either case, processing continues with element 224 as discussed herein below.

Once valid data has been entered as determined by element 214, or appropriate annotations are provided by elements 220 or 222, element 224 is next operable to determine whether the clinician has indicated that the data fields entered in the form are completed and therefore ready for submission to further processing. If the form is not yet complete, processing continues at element 204 to await further input from the clinician. If element 224 determines that the clinician has indicated completion of the form by requesting submission thereof, element 226 is next operable to generate a client certificate to secure the submitted data from unauthorized access. As noted above, numerous security techniques and structures may be implied to provide the desired security from unauthorized access. Digital data encryption utilizing public or private keys or so called client certificates are but exemplary of such security measures.

Element 228 is then operable to store the entered form data and any associated digitized images data in a secure local store associated with the remote computing device. As noted above, the secure local store is secured from unauthorized access through the client certificate or other security and encryption techniques as discussed above. The data so stored may be forwarded to the central server for permanent storage when communication capabilities allow such transmission. Element 230 is therefore operable to determine whether the central server is presently connected to the remote computing device. If not, processing of the method completes with the entered form data and associated digitized images data stored in the secure local store of the remote computing device. The central server may be temporarily inaccessible due to a failure of the communication link or communication medium or, as in the case of wireless communications, temporarily out of range. If element 230 determines that the central server is presently connected or if the connection is later restored (i.e., a wireless connection brought it back in range), element 232 is next operable to forward the locally stored data to the central server. Central server may provide a permanent archive of the clinical trial data as well as security features to preclude unauthorized access thereto. Since the central server may provide such a permanent archival storage, the remote computing device may remove its locally stored copy of the forwarded clinical trial data. Well known communication techniques and protocols may be employed to verify proper receipt of the forwarded clinical trial data such that it may be erased from the remote computing device upon verification of proper receipt. In addition, features of the central server may provide further validation of the forwarded clinical trial data. If an error is detected from such additional validation, the remote computing device may be so notified to permit corrective action by the clinician at the remote computing device. Alternatively, such corrective actions may be provided by users of the central server.

Those of ordinary skill in the art will recognize a variety of equivalent processes and steps to implement features and aspects hereof. The process of FIG. 2 is therefore intended merely as exemplary of one such process for providing features and aspects hereof.

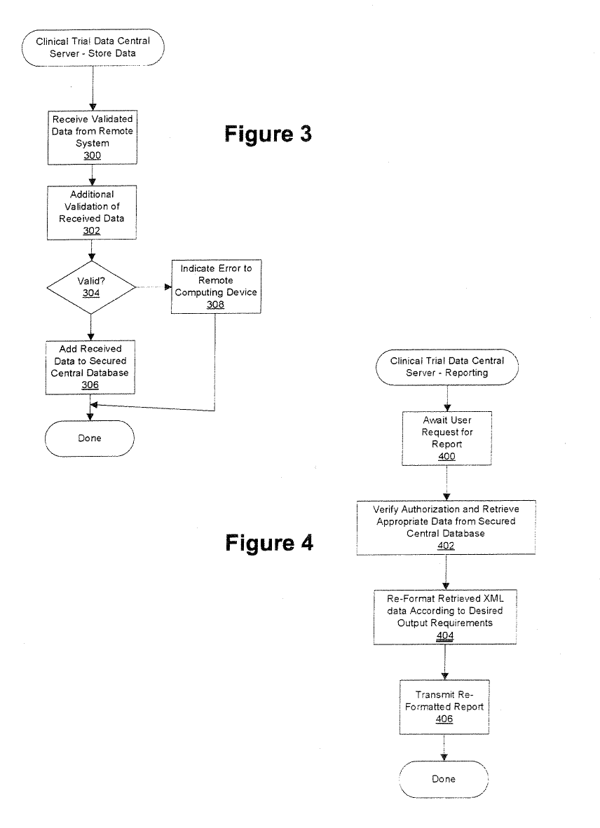

FIGS. 3 and 4 describe processes operable on a central server that provides centralized, permanent archive storage of clinical trial data as well as reporting and analysis tools therefore. In particular, FIG. 3 represents a process for receiving validated data from a remote computing device for persistent storage in the central server. Element 300 is first operable to receive such validated data from an associated remote computing device. Element 302 is next operable to further validate the received data. Although validation may be performed within the remote computing device as discussed above, further validation may be provided by the central server in view of its centralized store of other clinical trial data. For example, validation within the central server may include processing to detect duplicate entries for a particular patient or other anomalous or unauthorized data entry requests. If element 304 determines that the received data passes such a further validation, element 306 is then operable to add the newly received data to the secure central database of the central server. If element 304 determines that the received data fails to pass the additional validation, element 308 is operable to return an appropriate error indication to the remote computing device to permit further processing by the clinician. As noted above, such corrective actions may also be provided by users of the central system to avoid returning the invalid data to the clinician.

FIG. 4 is a flowchart describing a process operable within the central server for generating requested reports or analysis based on information saved in the secure central store associated with the central server. Element 400 awaits a user request for generation of an identified report or analysis. Element 402 then retrieves all appropriate data from the secured central database required to generate the identified report. Appropriate security measures may be employed to preclude unauthorized access by the requesting user. Element 404 then reformats the retrieved data according to the desired report requirements.

Data stored in and retrieved from the central database may be stored and retrieved as XML data messages allowing simple yet flexible reformatting capabilities. Element 406 transmits the reformatted report information to the requesting user or an appropriate regulatory agency as appropriate to the particular application.

Those of ordinary skill in the art will recognize a wide variety of similar processes operable within a central server to receive and store data from a remote computing device and to generate requested analysis reports therefrom. FIGS. 3 and 4 therefore represent merely exemplary processes for achieving desired features and aspects hereof.

While the invention has been illustrated and described in the drawings and foregoing description, such illustration and description is to be considered as exemplary and not restrictive in character. One embodiment of the invention and minor variants thereof have been shown and described. Protection is desired for all changes and modifications that come within the spirit of the invention. Those skilled in the art will appreciate variations of the abovedescribed embodiments that fall within the scope of the invention. As a result, the invention is not limited to the specific examples and illustrations discussed above, but only by the following claims and their equivalents.

1. A method for clinical trial data management comprising:
entering clinical trial data directly by an examining clinician using a remote computing device;
determining whether the remote computing device is communicatively coupled to a central server;
transmitting the clinical trial data from the remote computing device to the central server in response to a determination that the remote computing device is communicatively coupled to the central server; and
storing the data in a secure store within the remote computing device in response to a determination that the remote computing device is not communicatively coupled to the central server.

2. The method of claim 1 wherein the step of entering includes:
digitizing an image of hand drawn information entered by the clinician on the remote computing device.

3. The method of claim 1 wherein the step of entering includes:
entering fields of the clinical trial data in response to an electronic form presented to the clinician on the remote computing device.

4. The method of claim 3 wherein the step of entering form data further includes:
validating the entered clinical trial data within the remote computing device.

5. The method of claim 4 wherein the electronic form is defined as an XML schema and wherein the step of validating comprises validating the entered clinical trial data according to the XML schema.

6. The method of claim 5 wherein the step of validating further comprises:
comparing entered data against a corresponding edit specification in the XML schema; and
receiving clinician input in response to a determination that the entered data does not conform to the corresponding edit specification.

7. The method of claim 6 wherein the step of receiving clinician input includes:
receiving corrected data that adheres to the corresponding edit specification.

8. The method of claim 6 wherein the step of receiving clinician input includes:
receiving validation annotation information recorded with the entered data to explain the mis-comparison of the entered data relative to the corresponding edit specification.

9. The method of claim 6 wherein the step of receiving clinician input includes:
receiving validation status information recorded with the entered data to indicate the status of further investigation regarding the mis-comparison of the entered data relative to the corresponding edit specification.

10. The method of claim 1 wherein the step of storing includes:
employing a client certificate to secure the clinic trial data in the secure store of the remote computing system; and
associating the client certificate with the clinical trial data to secure the clinical trial data from unauthorized access.

11. A method of clinical trial data management comprising:
receiving clinical trial data from an examining clinician using a remote computer system;
determining whether the remote computer system is presently communicatively coupled to a central system;
forwarding the clinical trial data as XML messages to the central system in response to a determination that the remote computer system is presently coupled to the central system;
spooling the clinical trial data on the remote computer system in response to a determination that the remote computer system is not presently coupled to the central system wherein spooling includes securing the clinical trial data on the remote computer system to preclude unauthorized access thereto; and
transmitting the spooled clinical trial data to the central system as XML messages in response to a subsequent determination that the remote computer system is again coupled to the central system.

12. A system for clinical trial data management comprising:
a remote computer system for use by an examining clinician wherein the remote computer system includes:
form data entry components for presenting a form to the clinician and for receiving clinical trial data as responses to the presented form;
hand drawn data entry components to receive digitized images of hand drawn information as clinical trial data;
a remote communication link for coupling the remote computer system to a central system; and
a remote secured store for temporary storage of the clinical trial data until transmission of the clinical trial data to the central system may be accomplished;
a central system for centralized storage, analysis and reporting of the clinical trial data wherein the central system includes:
a central communication link for coupling the central system to the remote computer system; and
a central secured store for storage of the clinical trial data received from the remote computer system; and
a communication medium coupled to the remote communication link and coupled to the central communication link for exchanging the clinical trial data between the remote computer system and the central system.

13. The system of claim 12 wherein the remote computer system comprises a hand held computing device.

14. The system of claim 13 wherein the hand held computing device comprises a tablet PC.

15. The system of claim 13 wherein the hand held computing device comprises a laptop computer.

16. The system of claim 12 wherein the remote computer system comprises a desktop computing device.

17. The system of claim 12 wherein the communication medium comprises a wireless communication medium.

18. The system of claim 12 wherein the clinical trial data comprises XML messages.

19. A method in a computer system for managing clinical trial data entered by an examining clinician, comprising:
presenting to an examining clinician an electronic clinician data entry form defined as an XML schema on a remote display device of a remote computing system, said form having a set of data entry fields designed for receiving entry of a set of clinical trial data to be input by the examining clinician;
presenting to the examining clinician a prompt for selecting an option for manual entry of information; and
presenting to the examining clinician an indicator to begin manual entry of the digital information.

20. The method for managing clinical trial data as recited in claim 19 where presenting a prompt for selecting the option for manual entry further comprises presenting an option for manual entry utilizing a digital paper document scanner or manual entry utilizing an electronic digitizing tablet.

21. The method for managing clinical trial data as recited in claim 19, further comprising the steps of:
presenting to the examining clinician an invalid form data field entry indicator responsive to a validation processing computing component residing on the remote computing system and operable to compare clinician entered data with a corresponding edit specification in the XML schema and presenting to the examining clinician a reentry prompt where the clinician can select to reenter corrected form data; and
presenting a reentry field responsive to a selection of reentry.

22. The method of claim 21, where presenting the reentry prompt further comprises presenting an optional annotation prompt in the alternative for entry of annotated entered data with entered explanation why the entry flagged as invalid is acceptable; and presenting an annotated reentry field responsive to a selection of annotated reentry.

23. The method for managing clinical trial data as recited in claim 19, further comprising the steps:
presenting to the examining clinician a submission prompt for selection to indicate that data entry is completed and submission is requested.

24. The method of claim 23, further comprising the steps of:
presenting to a requesting user a report prompt for selecting generation of a report of entered clinical trial data; and
presenting to the requesting user responsive to a selection of the report prompt, a report of the appropriate clinical trial data reformatted from the XML data according to the output requirements corresponding to the report prompt selected.

25. The method of claim 24, where the report is reformatted from the XML data in accordance with requirements of a select regulatory agency corresponding to the report prompt selected.

26. A system for clinical trial data management comprising:
a remote computer system for use by an examining clinician wherein the remote computer system includes:
form data entry components for presenting a form to the clinician and for receiving clinical trial data as responses to the presented form;
hand drawn data entry components to receive digitized images of hand drawn information as clinical trial data;
a remote communication link for coupling the remote computer system to a central system; and
a remote secured store for temporary storage of the clinical trial data until transmission of the clinical trial data to the central system may be accomplished.

27. The system of claim 26, where the hand drawn data entry component is operable to receive digitized hand drawn images from a paper document scanning device communicably linked to the remote computer system and from an electronic digitizing tablet device communicably linked to the remote computer system.

28. The system of claim 26, where the remote computer system further comprises data entry validation components operable for validating data entered by the clinician by comparing entered data against a corresponding edit specification and for notifying the clinician when the data entry is invalid.

29. The system of claim 28, where the data entry validation component is further operable to prompt the clinician for reentry of corrected data and receive said corrected data and further operable to prompt the clinician to alternatively enter annotated data explaining why data need not be corrected and operable to receive said annotated data.

30. The system of claim 28, where the remote computer system further comprises:
a report selection component operable to receive a report selection from the clinician and further operable to initiate retrieval of the appropriate re-formatted XML clinical data responsive to and in accordance with the report selection.

31. The system of claim 30, where the remote computer system comprises a hand held computing device.

32. The system of claim 31 wherein the hand held computing device comprises a tablet PC.

33. The system of claim 31 wherein the hand held computing device comprises a laptop computer.

34. The system of claim 30 wherein the remote computer system comprises a desktop computing device.

35. The system of claim 26, further comprising:
a central system for centralized storage, analysis and reporting of the clinical trial data wherein the central system includes:
a central communication link for coupling the central system to the remote computer system;
a central secured store for storage of the clinical trial data received from the remote computer system; and
a communication medium coupled to the remote communication link and coupled to the central communication link for exchanging the clinical trial data between the remote computer system and the central system.

36. The system of claim 35 wherein the communication medium comprises a wireless communication medium.

37. The system of claim 35, where the central system further comprises:
a central data entry validation component operable to notify the remote computer system with a data invalidation error when data is determined to be invalid.

38. The system of claim 26, where the remote computer system further comprises:
a client certificate component operable to generate a client certificate to secure submitted data from unauthorized assets.

39. The system of claim 38, where the client certificate component is combined with a data encryption technique.
